(19) United States
(12) Patent Application Publication
(10) Pub. No.: US 2014/0194715 A1
(43) Pub. Date: Jul. 10, 2014

(54) Glucose Analyzing Blood Examiner

(71) Applicant: Case Western Reserve University, Cleveland, OH (US)

(72) Inventor: Mark Griswold, Shaker Heights, OH (US)

(21) Appl. No.: 14/205,483

(22) Filed: Mar. 12, 2014

Related U.S. Application Data
(63) Continuation-in-part of application No. 13/017,126, filed on Jan. 31, 2011.

(51) Int. Cl. A61B 5/145; G01R 33/48; A61M 5/172

(52) U.S. Cl. CPC A61B 5/14532; A61M 5/1723; G01R 33/4828
USPC 600/365; 604/67; 604/504; 600/309

(57) ABSTRACT

In one embodiment, a miniaturized wearable nuclear magnetic resonance (NMR) apparatus is described. The example NMR apparatus accesses data (e.g., table, mathematical expression) that describes a relation between a nuclear magnetic resonance (NMR) signal decay rate and a known concentration of glucose in a fluid. The NMR apparatus acquires, non-invasively and in-vivo, an observed NMR signal decay rate from a fluid in a patient, and estimates a concentration of glucose in the fluid in the patient by comparing the observed NMR signal decay rate with the data that describes the relation between the NMR signal decay rate and the known concentration of glucose. The data may be generic to a population and a class of devices or may be customized to an individual patient and an individual device.

### CROSS REFERENCE TO RELATED APPLICATIONS

This application is a continuation-in-part of U.S. patent application Ser. No. 13/017,126 filed Jan. 31, 2011.

### BACKGROUND

According to NIH Publication No. 99-4398, in 1999, diabetes affected an estimated 16 million Americans. As of 1999, about 800,000 new cases were diagnosed annually. In 1999, diabetes was the sixth leading cause of death due to disease in the United States. Since 1980, the age-adjusted death rate due to diabetes has increased by 30 percent. Over the same time period the death rate has decreased for other common multifactorial diseases (e.g., cardiovascular disease, stroke). In 1999, the cost of diabetes to the United States was over $105 billion. More than one out of every ten U.S. health care dollars was spent for diabetes. About one out of every four Medicare dollars was spent on health care for people with diabetes.

Diabetes Mellitus, which is commonly referred to more concisely as diabetes, is a chronic disease that affects the ability of the body to maintain desired blood sugar levels. Type 1 diabetes occurs when the pancreas is unable to produce insulin in amounts sufficient to properly control blood sugar levels. Type 1 diabetes may occur when the body actually attacks and destroys cells that are supposed to produce insulin. Thus, type 1 diabetes is considered to be an autoimmune disease in which unknown environmental factors combine with genetic susceptibility to destroy pancreatic beta cells that produce insulin in healthy humans.

The pancreas is supposed to produce two hormones that together act to maintain desired blood sugar levels. Insulin is supposed to be produced when blood glucose levels get too high. Insulin instructs the body's cells to take in glucose from the blood. Glucagon is supposed to be released when blood glucose levels start to fall too low. Glucagon instructs the liver to convert stored glycogen into glucose and release it into the bloodstream. The action of glucagon is thus opposite to that of insulin. Glucagon also stimulates the release of insulin, so that newly-available glucose in the bloodstream can be taken up and used by insulin-dependent tissues. A diabetic pancreas does not produce appropriate hormones in appropriate amounts at appropriate times, and thus, blood glucose levels can reach undesired levels. Thus, the key characteristic of type 1 diabetes is the inability to manufacture desired amounts of insulin.

Even though the causes are different, a similar set of dysfunctions occur with Type II diabetes. In Type II diabetes, the cells of the body are unable to receive or transport the sugars from the blood stream into the cells, again leading to a lack of control of blood glucose concentration.

Undesired blood glucose levels can include too much sugar in the blood (hyperglycemia) and too little sugar in the blood (hypoglycemia). Eating can increase blood sugar levels. Insulin can lower blood sugar levels. Therefore, all people with type 1 diabetes and some with type II take insulin.

Historically, insulin has been injected under the skin. To achieve the goal of maintaining blood sugar levels in a desired range, diabetics will frequently test their blood sugar levels. Based on the measured blood sugar level, diabetics will ideally inject an appropriate amount and type of insulin at an appropriate time.

Recently, insulin pumps have been used to deliver insulin continuously and/or periodically. Insulin pumps may be programmed to deliver insulin in set amounts at set times. Some insulin pumps may even be programmed to deliver different amounts on demand based on blood sugar level measurements.

Determining insulin dosage and delivery time depends on accurately measuring blood sugar levels. Conventionally, measuring blood sugar levels has been performed by chemically analyzing blood or interstitial fluids. Chemically analyzing blood has conventionally required access to blood and then sacrificing the accessed blood during the chemical test. To acquire the blood a diabetic may prick their finger to get a blood drop. The diabetic will then put the drop of blood in a glucose meter and read the measurement. Alternatively, a probe (e.g., catheter) may be inserted into the body to maintain substantially constant access to a blood or interstitial fluid source.

Both the finger prick technique and the inserted probe technique are invasive, can be painful, and can provide entry points for bacteria, virus, and infection. Furthermore, since both techniques involve a chemical reaction, the measurements may not always be as accurate as desired. Also, when the diabetic has to read the measurement from the device, there is an opportunity for the measurement to be misread. These inaccuracies can lead to inappropriate amounts of insulin being delivered at inappropriate times.

Issues associated with inaccurate blood sugar level measurements are exacerbated by the fact that different insulin preparations have different characteristics. For example, different insulin preparations may begin working at different times, may work at different rates, and may work for different periods of time. Making things even worse, different insulin preparations may act differently under different conditions (e.g., age of preparation, exposure to heat, exposure to other chemicals). Conventionally it may have been difficult for the diabetic, especially under certain conditions (e.g., stress, shock) to compute proper dosages and/or mixtures. Thus, it becomes even more likely that inappropriate amounts of insulin can be delivered at inappropriate times. Even if accurate measurements were taken and accurate dosages computed, the actual effect of the injected insulin can only be analyzed by a subsequent measurement.

A diabetic may experience different insulin demands from day to day, and even from moment to moment as a function of activity, stress, environmental factors, and so on. Therefore, the diabetic may need to be provided with different amounts of insulin when exercising, when sick, when eating more or less than normal, when travelling, when under stress, and under other conditions. However, the conditions that produce the varying needs may also make it difficult, if even possible at all, to make an accurate blood sugar level measurement. Particularly difficult conditions for monitoring blood sugar levels and delivering appropriate amounts of insulin include during surgery, during high intensity exercise, while asleep, while in a coma, during childbirth, while suffering from dementia, while suffering from Alzheimer's, and so on.

Therefore, attempts are constantly being made to improve blood sugar level measurement techniques and accuracy and to identify feedback mechanisms to facilitate delivering appropriate amounts of insulin at appropriate times.

### BRIEF DESCRIPTION OF THE DRAWINGS

The accompanying drawings, which are incorporated in and constitute a part of the specification, illustrate various example systems, methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 illustrates an embodiment of an NMR apparatus to measure an amount of a chemical species in a sample.

FIG. 2 illustrates an embodiment of an NMR apparatus configured with therapeutic elements.

FIG. 3 illustrates an embodiment of an NMR apparatus configured with drug delivery elements.

FIG. 4 illustrates an embodiment of an NMR apparatus configured with a second field generating apparatus.

FIG. 5 illustrates a method associated with measuring an amount of chemical species in a sample associated with an NMR apparatus.

FIG. 6 illustrates a method associated with measuring an amount of chemical species in a sample associated with an NMR apparatus.

FIG. 7 illustrates a single signal decay curve with a glucose concentration at 100 mg/dl.

FIG. 8 illustrates the decay rate of an MR signal (T2eff) over multiple measurements in diluted fetal bovine serum (FBS) with various levels of glucose.

FIG. 9 illustrates the high linear correlation between T2eff and glucose concentration across the physiological range.

FIG. 10 illustrates representative signal decay curves for pure water and oil.

### DETAILED DESCRIPTION

The diffusion speed of water in interstitial fluid varies with the concentration of glucose in the water. The decay rate of an NMR signal produced by water in the interstitial fluid also varies with the concentration of glucose in the water. The concentration of glucose in the water affects the viscosity of the water, which in turn affects the decay rate of the NMR signal. A device, that measures the decay rate of an NMR signal can be calibrated to actual glucose concentration measurements that are acquired using a different glucose concentration apparatus to facilitate estimating glucose concentrations from observed NMR signal decay rates. Observed NMR signal decay rates can be used to access calibration data that identifies known glucose concentrations. Calibration data for glucose, or for other chemical species, can be acquired, stored, and made available for example apparatus and methods to access.

Example apparatus and methods provide a new class of glucose sensing technology based on magnetic resonance (MR). Example MR-based sensing does not detect glucose directly. Instead, example MR-based sensing detects the effect of glucose on the water in the interstitial fluid of a patient. Fluids with sugar in them are “sticky”, meaning they have a higher viscosity. For more than 50 years it has been clearly established that the rate of viscosity can be directly measured and thus the diffusion of water at the molecular level can be measured using MR techniques. See, for example, R. Freedman, et al. Wettability, Saturation, and Viscosity From NMR Measurements. December 2003 Society of Petroleum Engineers Journal, and R. DeLaPaz et al, ACR Appropriateness Criteria on Cerebrovascular Disease, Journal of the American College of Radiology, 8(8), August 2011, Pages 532-538.

Example apparatus and methods adapt this MR-based technology to the non-invasive estimation of glucose concentration in vivo using a miniaturized, wearable MR system. Example apparatus and methods provide multiple advantages over conventional systems. First, the MR-based technology is completely non-invasive. It will sit on the skin, and will not physically penetrate the skin in any way. In addition, the method has a long, established safety record in the clinic, and has no known side effects. Another advantage of MR-based methods is that they are based on sensing water, not glucose directly. Because of this, these methods have essentially the same, linear sensitivity throughout the physiological range of glucose. There is no saturation of glucose at the high end of the physiological range, nor loss of sensitivity even with no glucose present. Because of this, there is no possibility of confusing a device failure with a very low glucose reading.

The concept behind the MR-based sensing is not to detect the glucose directly, but to detect its effect on the water in the interstitial fluid. This effect is actually something that we all know about through our daily lives—fluids with sugar in them, such as sodas and juices, are “sticky” or “thick.” The physics term for this is that the viscosity is higher, meaning that the water molecules in the fluid have a harder time moving around in the presence of other molecules like sugars. Albert Einstein proposed this link as early as 1905, which is now formalized in the Einstein-Stokes equation, which links viscosity to the rate of diffusion of a molecule. The Stokes-Einstein equation is the equation first derived by Einstein in his Ph.D thesis for the diffusion coefficient of a “Stokes” particle undergoing Brownian Motion in a quiescent fluid at uniform temperature. The result was formerly published in Einstein's (1905) classic paper on the theory of Brownian motion. Einstein's result for the diffusion coefficient D of a spherical particle of radius a in a fluid of dynamic viscosity h at absolute temperature T is:

$$D = \frac{\overline{R} T}{{N}_{A}} \frac{1}{6 π η a}$$

where R̅ is the gas constant and N_{A} is Avogadro's Number. Thus when there is little glucose present, the diffusion rate of water will be higher compared to when there is more glucose. Said another way, the diffusion rate of water varies inversely with the concentration of glucose. While this relationship has been known, it has been impossible to non-invasively measure the diffusion rate of water in interstitial fluid using a miniaturized (e.g., wearable, portable) device until the introduction of example apparatus and methods.

The fundamental concept of the glucose sensing technology is that the rate of decay of the MR signal from the sample is proportional to the viscosity of the fluid under investigation. See, for example, D. Rata et al, Self-diffusion measurements by a mobile single-sided NMR sensor with improved magnetic field gradient. Journal of Magnetic Resonance 180(2), June 2006, Pages 229-235 Example apparatus and methods rely on the fact that the viscosity of the interstitial fluid is proportional to the concentration of glucose and that the decay rate of the MR signal varies with viscosity. Thus, measuring the decay rate of the MR signal from the interstitial fluid provides data from which glucose concentration can be determined by comparing the decay rate to known (decay rate, glucose concentration) pairs found in calibration data.

Example apparatus and methods rely upon magnets that are significantly smaller than magnets in conventional systems but which are comparably strong. The systems will not generate a signal from the whole body but only from a superficial location just below the surface of the skin where the device is placed. In one embodiment, a device may be 2.5 cm×2.5 cm×1.5cm in size and weigh less than a 1 kg. In one embodiment, the MR-based device is entirely self-contained and requires no disposable parts. An example device can easily be removed, and will therefore not interfere in other medical procedures, especially medical imaging procedures (e.g., x-ray, MRI, CT, ultrasound). This contrasts directly with any device that requires implantation into the body. Besides inducing inflammation other immune responses, invasive or implantable glucose monitoring devices can interfere in all of these medical imaging procedures, and may even be a contraindication for some of them, especially MRI.

Example MR-based methods are based on sensing the effect of glucose on water, not glucose directly. Because of this, example methods have essentially the same, linear sensitivity throughout the physiological range of glucose. There is no saturation of glucose at the high end of the physiological range, nor loss of sensitivity even with no glucose present. Because of this, there is no possibility of confusing a device failure with a very low glucose reading. There is either a signal from the water, which has a specific known form, or there isn't.

Magnetic resonance (MR) is analogous to striking a bell. When the bell is struck it produces a sound that slowly decays away. In much the same way, the MR signal in an interstitial fluid decays away exponentially after excitation, The time constant of this decay after excitation in MR is called T2. T2 values are consistent for a given tissue within a very narrow range across all humans. This reproducibility forms the basis of much of clinical MRI. In a general sense, the T2 value describes how fluid a given tissue is, which is related, at least in part, to its average viscosity. MR allows choosing how different diffusion rates or viscosities affect T2 through the choice of the RF pulse excitation patterns used in a given experiment. For example, RF pulse excitations can be chosen so that the MR signal persists for a long time (e.g., long T2) in a tissue or solution with a low viscosity (e.g., high diffusion rate), such as pure water, while the signal decays rapidly for a high viscosity, dense tissue, such as white matter (e.g., shorter T2). Alternatively, RF pulse excitations can be chosen to establish the opposite situation, where the signal decays faster in high diffusion tissues.

Clinical MR relies on the fact that there are multiple physiological effects that cause a change in the observed T2 and potentially also viscosity, such as changes in cell density, vascularization, edema, or other factors. Thus, changes in absolute T2 or viscosity may not be specific to changes in glucose. Therefore, example apparatus and methods rely on the fact that changes in glucose that are relevant to substantially constant glucose measuring occur on a significantly faster time scale than other physiological changes that may affect T2 or average viscosity. This fact is particularly true for patients with type-1 diabetes where glucose levels can change on the time scale of minutes, while changes due to other causes (e.g., cellular density, edema) change on a relatively slower time scale.

Example apparatus and methods may employ a conventional glucometer to provide measurements to calibrate the T2 measurements. This calibration provides a specific link between a measured T2 rate and the interstitial glucose values that are determined by the decay rate of the MR signal for the patient. For example, T2 decay rates for a patient or for a population can be measured at different known glucose concentrations. The known glucose concentrations may be measured with conventional techniques for measuring the concentration of a chemical species in water. The T2 decay rates associated with the different known concentrations can be stored. Later observed T2 decay rates can then be used to estimate glucose concentrations by referencing the stored data that relates T2 decay rates to known concentrations.

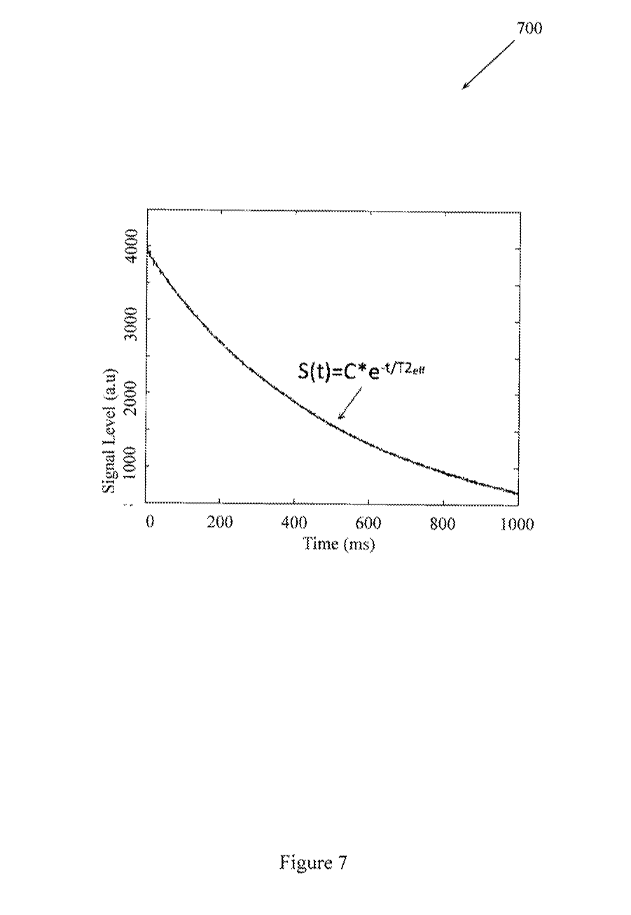

FIGS. 7, 8, and 9 illustrate results demonstrating multiple measurements across different samples of 4× diluted fetal bovine serum (FBS) with various levels of glucose acquired in rapid succession. FIG. 7 illustrates a single signal decay curve with a glucose concentration at 100 mg/dl. In one embodiment, the MR signal S may be modelled by:

S(t)=C*e^{-t/T}2eff
where C is a constant that accounts for multiplicative factors including, but not limited to, coil sensitivity, and receiver gain, and where e is the exponential constant.

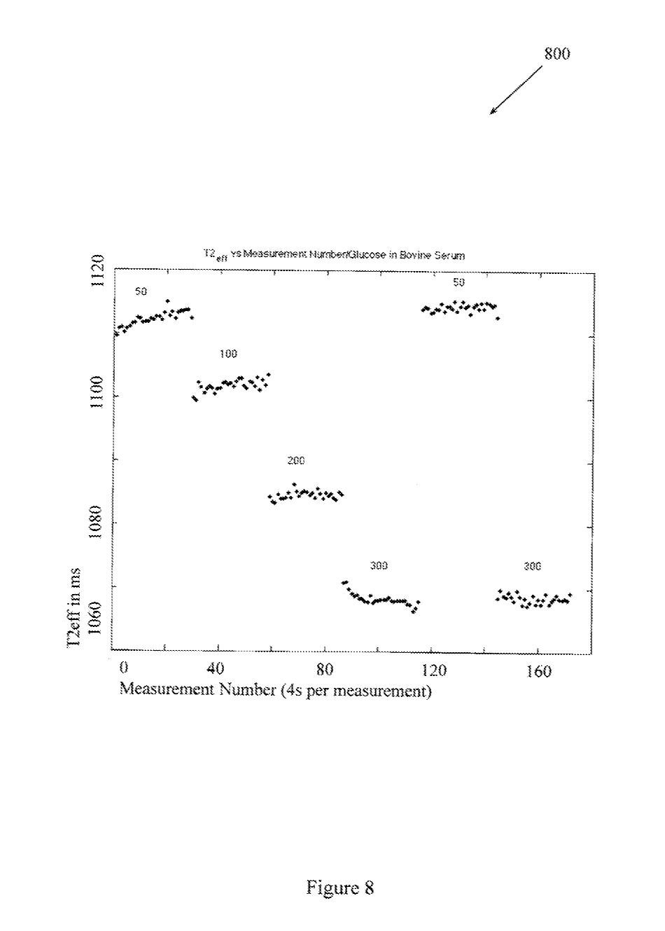

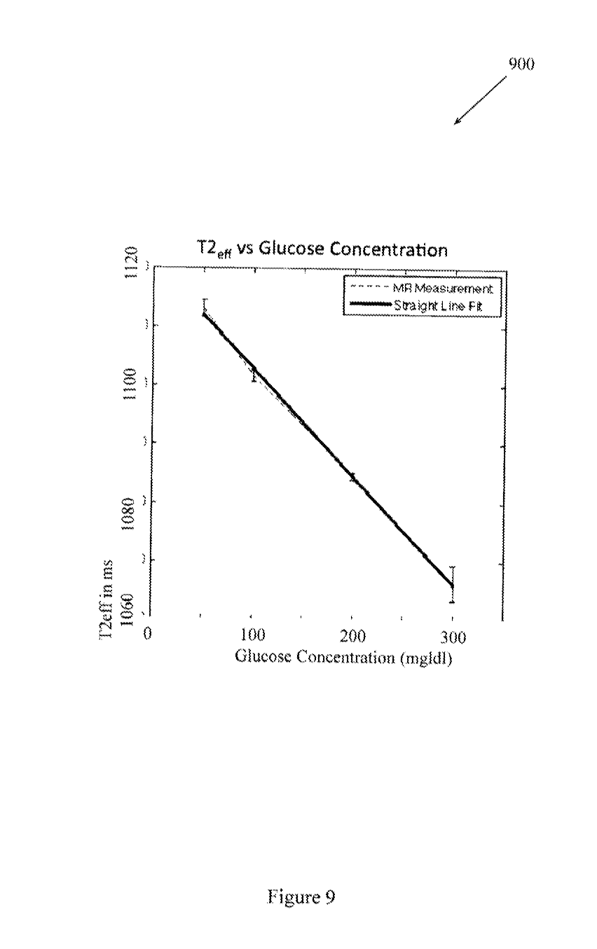

FIG. 8 illustrates the decay rate of an MR signal (T2_{eff}) over multiple measurements in diluted fetal bovine serum (FBS) with various levels of glucose. FIG. 9 illustrates the high linear correlation between T2_{eff} and glucose concentration across the physiological range.

FIG. 7 illustrates a representative single measurement. Data were fit to determine the effective T2 (T2_{eff}). Data points were acquired in 1 s with a 3 s delay to the next measurement. As can be seen in FIG. 9, the data show that the measurement of T2_{eff} is reproducible and shows a high linear correlation to glucose concentration across the physiological range of glucose concentrations. Since the T2 is reproducible and linearly correlated to glucose concentration, calibration data acquired with conventional glucose concentration measuring apparatus may be stored to facilitate estimating glucose concentrations from later observed T2 decay rates. The standard deviation of these measurements is ˜1.4 mg/dl per 4 s acquisition time. In one example, a wearable device may acquire a measurement every 5 minutes. Example systems may accept +/−20 mg/dl standard deviation of errors in glucose estimated from the decay of T2_{eff} in the interstitial fluid.

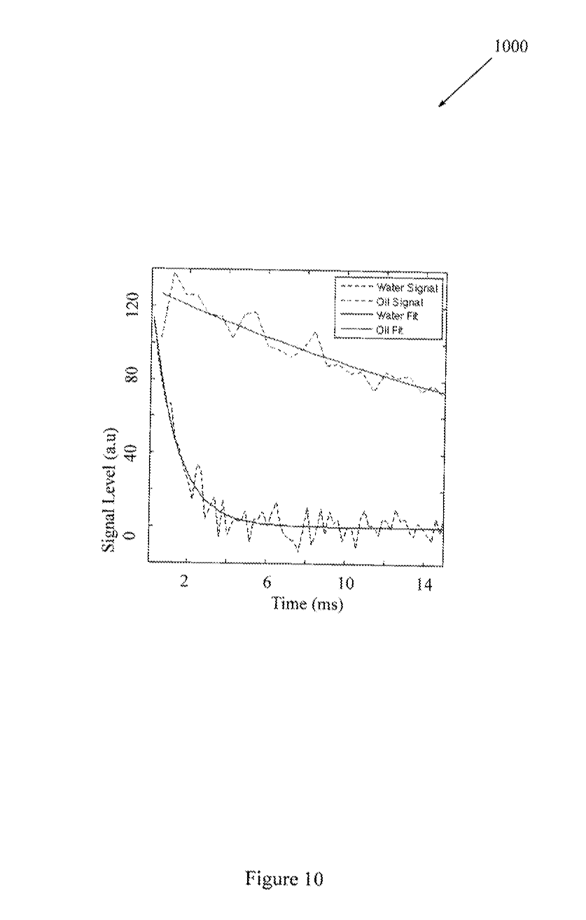

An initial prototype scale model wearable magnet system was developed. In the prototype, the magnet is approximately 4×4×4 cm³ in size. Experiments performed on oil and water were conducted using the prototype. The experiments observed the significant differences in diffusion/viscosity illustrated in FIG. 10. One example wearable MR device includes a magnet, an antenna, an RF transmitter, an RE receiver, a controller, and a battery. The recent dramatic advances in cellular telephones and computers have opened the door for truly miniaturized antennas, transmitters, receivers, controllers and batteries. Most of us carry around phones with more computing and communication capabilities than a complete desktop computer a decade ago. A similar reduction in size and increase in computational power may be applied to MR-based electronics for example apparatus.

Example systems and methods non-invasively and accurately measure blood sugar glucose levels substantially constantly and substantially in real-time based on correlations between T2 decay rate and glucose concentration established from calibration data. Example systems and methods employ diffusion nuclear magnetic resonance (NMR) to non-invasively determine blood glucose levels as a function of T2 decay as influenced by water viscosity as determined by glucose concentration. Blood cells, other cells, and fluid in intercellular spaces will produce different NMR signals as a function of different blood glucose levels. Diffusion speed in the interstitial fluid is affected by the amount of glucose in the fluid. Diffusion speed correlates with T2 decay.

Envision a twelve ounce glass filled with six ounces of pure water. A T2 decay or other NMR signal decay associated with the pure water can be observed and recorded. Now envision adding one tablespoon of sugar to the water. Adding the tablespoon of sugar to the water changes the water. A T2 decay or other NMR signal decay associated with the known concentration of sugar in the water can be observed and recorded. Now envision repeatedly changing the known concentration of sugar in the water and acquiring T2 or other NMR signal decay data associated with the known concentrations. Understandable properties of the water will change as the amount of sugar in the water changes. For example, the viscosity of the water will change. Similarly, the transparency of the water may change. As described and quantified by Einstein, the amount of a chemical (e.g., glucose) in a fluid affects diffusion. The higher the glucose level, the lower the diffusion of the water through the solution. This effect is observable using NMR techniques. Measurements of glucose concentrations using conventional systems can be calibrated to MR signal decay rates and then subsequent MR signal decay rates can be used to estimate glucose concentrations. In one embodiment, MR signal decay rates may be calibrated on a per patient basis. In one embodiment, MR signal decay rates may be calibrated on a population basis.

Example systems and methods facilitate acquiring NMR signals from the water and monitoring their decay rates under defined (e.g., known concentration) situations. The NMR signals accurately measure the amount of sugar in the water and/or the change in the amount of sugar in the water based on the decay rate of the MR signal. The decay rate of the MR signal varies inversely with the concentration of glucose in the water. The NMR signals are acquired without touching the water. Thus, example systems and methods do not require a finger stick or an embedded sensor that is in contact with blood to be able to measure sugar levels.

Example systems measure the decay of the NMR signal in the presence of either a switched or constant inhomogeneous applied magnetic field. Example systems may employ a constant inhomogeneous main magnetic field, or a gradient field. One example system measures the apparent NMR signal decay rate using an inhomogeneous permanent magnet as the polarizing field for the NMR experiment. Another example system performs conventional diffusion weighted NMR using pulsed or constant field gradients.

Conventionally, NMR systems have been room-sized. Therefore, conventionally it has been impractical to measure blood sugar levels for diabetics using an NMR system. It is impractical to live inside an MRI apparatus. Therefore, example systems provide miniaturized apparatus that produce very local conditions sufficient to perform very local NMR. Example methods employ the miniaturized apparatus to acquire NMR signals from interstitial fluid. The NMR signals are then analyzed to determine blood sugar levels by analyzing the decay rate of the MR signal. Analyzing the decay rate may involve referring to previously acquired calibration data. In one embodiment, the blood sugar levels determined from the NMR signals are then used to control an insulin pump.

NMR spectroscopy provides a non-destructive, quantitative analytical method. Many organic molecules have NMR-active nuclei. Diffusion constants for molecular sized objects in solution can be measured using NMR. The diffusion constants can be measured with an accuracy approaching 1%. Example systems and methods may employ diffusion NMR to measure blood glucose levels. Diffusion NMR separates mixture components spectrally based on differing translational diffusion coefficients of chemical species in solution. Therefore, example systems and methods acquire NMR diffusion measurements of complex samples including glucose and other chemicals found in the human body.

Example systems and methods may probe diffusion using pulsed field gradient NMR. Pulsed field gradient NMR applies magnetic gradient pulses to a sample located in a static magnetic field produced by the apparatus. The magnetic field generated by the gradient pulse varies across the sample. Therefore, molecules in one sample area are subjected to a different magnetic field than molecules in a different sample area. Therefore, the Larmor frequency of the molecules is different meaning that the gradient pulse phase encodes spins according to molecular position. After the gradient pulse, molecules may diffuse from one sample location to another sample location. After a period of time, a dephasing gradient is applied to reverse the phase change produced by the encoding gradient. If an encoded molecule has moved from one location to another, then it will not have its phase encoding reversed and will not be decoded.

A nearly identical effect occurs when the field gradient or inhomogeneity is constant or nearly constant in time. RF pulses are applied and the decay rate of the signal represents both the intrinsic relaxation of the molecule under investigation as well as additional relaxation due to diffusion. Thus, either system configuration can result in an accurate measurement of molecular motion or diffusion.

In one example, a sample is subjected to a radio frequency (RF) pulse. The sample includes fluid in which glucose or another chemical species may be present. The RF pulse turns the equilibrium magnetization M0 in the sample into the transverse plane, which is perpendicular to the main static magnetic field B0. The magnetization vector rotates around B0 at an angular frequency ω=dφ/dt given by the Larmor equation:

ω=γB0

where γ is the gyromagnetic ratio (γ=2π×42.576 rad s⁻¹T⁻¹ for a hydrogen proton).

After the initial excitation by the RF pulse, some additional magnetic field is present. This could take the form of an inhomogeneous main field or a gradient field G(x,y,z). For example, applying G(x) changes B0 to a spatially variable field B (x,y,z) B0+G(x,y,z) where G(x,y,z) is substantially non-zero over some fraction of the sensitive volume of the system. A similar formulation can be made in the case of an inhomogeneous main field. Because the field is now spatially varying, Larmor frequencies become different at different places in the sample. Thus, after some period of time, some phase differences may have accumulated between the spins at different positions.

After waiting for a diffusion time t=nΔt, an additional RF pulse is applied that flips the spins in the transverse plane. This causes the spins to start to rephase according to their spatial position in the field inhomogeneity. For spins that did not change positions during the diffusion time, the phase differences will be completely reversed. For spins that did change position during the diffusion time, they will not see exactly the same inhomogeneity, and thus, the spins will not exactly reverse phase difference from the first period. This incomplete reversal yields a phase dispersion in the measured sample. Faster diffusion means that the spins have more opportunity to travel farther and therefore experience larger magnetic field changes. Slower diffusion means that spins have less opportunity to travel and therefore experience smaller magnetic field changes. Diffusion speed in the interstitial fluid is affected by the amount of glucose or other chemical species in the fluid. Therefore, the described NMR methods can be used to measure blood glucose levels or the levels of other chemical species for which calibration data relating concentration to MR signal decay rate is available.

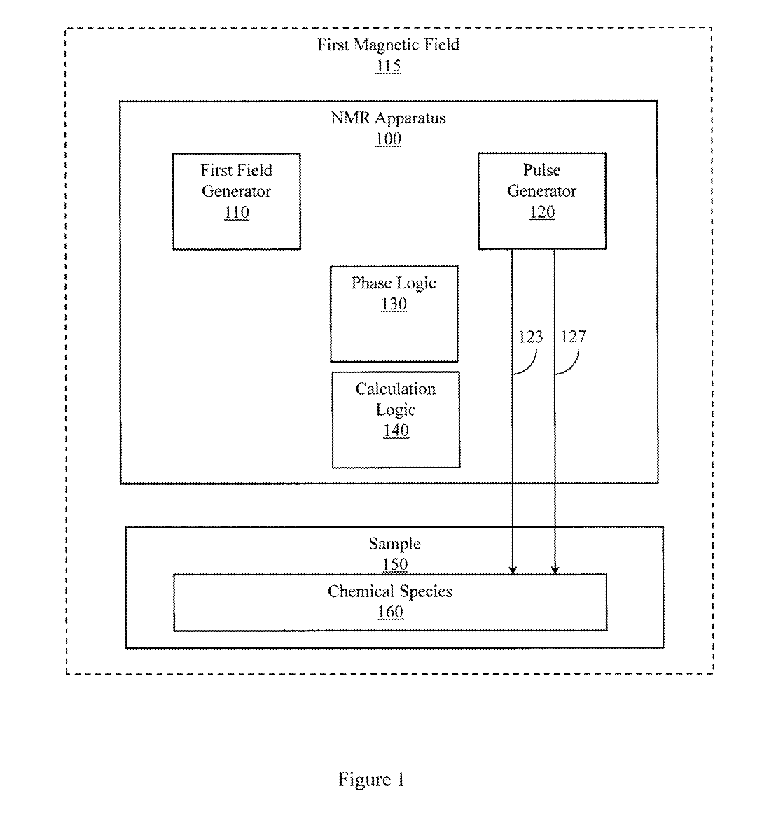

FIG. 1 illustrates an embodiment of a NMR apparatus 100 for determining the amount of a chemical species in a sample. The NMR apparatus includes a first field generator 110 that is configured to provide a first magnetic field 115 suitable for NMR. The first magnetic field 115 may be a static inhomogeneous applied magnetic field configured not to change in time. The first magnetic field 115 is sufficiently large to encompass a sample 150 or a region of interest of the sample 150.

A pulse generator 120 provides a first RF pulse sequence. The pulse generator 120 uses frequencies associated with NMR. The RF pulse sequence may include a first RF pulse 123 to excite nuclei associated with a chemical species 160 in the sample 150. The pulse sequence may also include a second RF pulse 127 to cause the nuclei of the chemical species 160 to rephase according to their spatial position in the first magnetic field 115. The frequency of the RF pulse sequence is chosen to produce an NMR signal associated with a specific chemical species 160 (e.g., glucose) in the sample 150 (e.g., blood, tissue, organ). The amount of the chemical species 160 in the sample 150 can be measured as a function of the decay of the NMR signal by relating the observed decay rate to previously acquired calibration data. A phase logic 130 is configured to measure the NMR signal decay.

The phase logic 130 measures NMR signal decay by measuring the phase differences that have accumulated between the spins of the nuclei of the chemical species 160 in the sample 150. The NMR signal may be used to discern information about the chemical species 150 in the sample 160 by referring to previously acquired data that relates decay rate to concentration. Since decay rates vary linearly and reproducibly with concentration, when a decay rate is observed for which an exact match is not available in the previously acquired calibration data, an estimate can be made by interpolating between known data points. In one embodiment, the previously acquired calibration data may be available as a table or other collection of related data points. In one embodiment the previously acquired calibration data may be available as a mathematical formula or expression. In one embodiment, the NMR signal decay represents, at least in part, the intrinsic relaxation of the chemical species 160. In another embodiment, the NMR signal decay may represent decay due to diffusion.

The sample 150 may be a interstitial fluid and the chemical species 160 may be glucose. Interstitial fluid is capable of diffusion in the body. Therefore, the phase logic 130 may be configured to measure NMR diffusion. In one embodiment, the pulse generator is configured to initially excite the nuclei of the chemical species 160 with a first RF pulse 123. The pulse generator is configured to apply a second RF pulse 127 to the sample 150. The second RE pulse 127 causes the spins of the nuclei of the chemical species 160 in the sample 150 to rephase based on their position in the first magnetic field 115.

A calculation logic 140 measures the amount of the chemical species 160 (e.g., glucose) in the sample 150 (e.g., interstitial fluid) by comparing the NMR signal decay to previously acquired data that relates concentration to NMR signal decay. If the nuclei of the glucose were able to travel through interstitial fluid, the glucose would experience larger magnetic field changes. Therefore, if the glucose experiences larger magnetic field changes, water in the interstitial fluid was able to diffuse more quickly indicating less glucose in the interstitial fluid. Conversely, if the glucose experiences fewer magnetic field changes, the water was not able to diffuse as quickly, indicating more of the glucose in the interstitial fluid.

Recall that conventional NMR systems have issues associated with size. The example apparatuses and methods do not require the use of conventional NMR systems. Instead, NMR 100 apparatus may be a miniaturized apparatus that produces very local conditions sufficient to perform very local NMR. For example, the first field generator 110 may be a small neodymium magnet used to generate a first magnetic field 115 that will not change in time. The pulse generator 120 provides an RF pulse sequence with an oscillation rate in a range of approximately 30 kHz to 300 GHz. pulse generator 120 capable of this oscillation rate may be very small and stamped on a small circuit board using surface mount technology (SMT) or through hole technology (THT) mounts In one embodiment, the phase logic 130 and a calculation logic 140 are implemented on a microprocessor. Accordingly, the NMR apparatus may be sufficiently small to be mobile and wearable.

In one embodiment, NMR apparatus 100 is mobile to be more practical for patients that may require constant measurements of a chemical species 160 in their bodies. For example, a diabetic patient may require substantially constant monitoring of glucose levels in the blood. A mobile NMR apparatus 100 may be wearable or implantable. Rather than incessant finger pricks or invasive probing (e.g. catheter), a mobile NMR device that could be worn (e.g. in manner of a watch, pendant) or implanted allows a patient a greater degree of freedom and convenience. Furthermore, a wearable or implantable NMR apparatus 100 does not introduce the risk of infection of its transdermal counterparts (e.g. finger prick, probe, catheter).

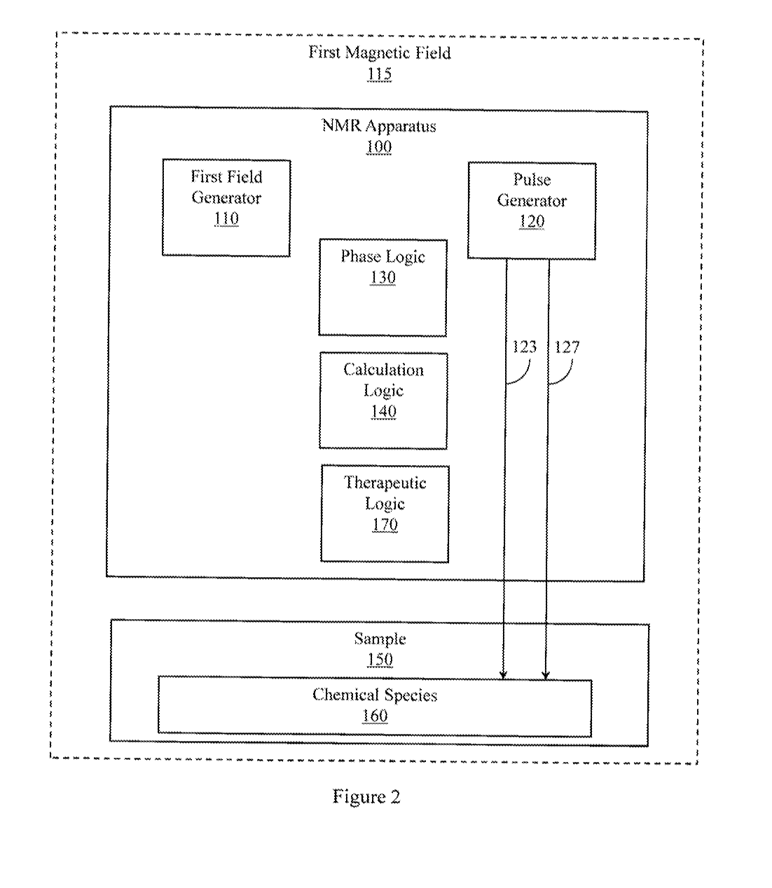

FIG. 2 illustrates an embodiment of NMR apparatus 100 that is configured with a therapeutic logic 170. In this embodiment, the therapeutic logic 170 determines an amount of insulin to be administered to a patient as a function of the measure of the amount of the chemical species 160 (e.g., glucose) in the sample 150 (e.g., interstitial fluid, blood).

In one embodiment, the calculation logic 140 may measure the amount of chemical species 160 in the sample 150 based on a diffusion rate that is determined to be low where the diffusion rate is computed with reference to previously observed MR signal decay rates at known concentrations. If the measurement of the chemical species 160 in the sample 150 is outside of a predetermined range, the therapeutic logic 170 may determine an amount of insulin to be administered to the patient corresponding to the measured amount of chemical species 160 in the sample 150.

Recall that there are several opportunities for error when a patient attempts to determine the correct amount of insulin to give self-administer. Therefore, the therapeutic logic 170 determining the correct amount of insulin for the patient, rather than having a patient calculate the amount of insulin, eliminates an opportunity for error. To further reduce the opportunity for error, the NMR apparatus 100 may include an insulin pump to administer the amount of insulin to the patient.

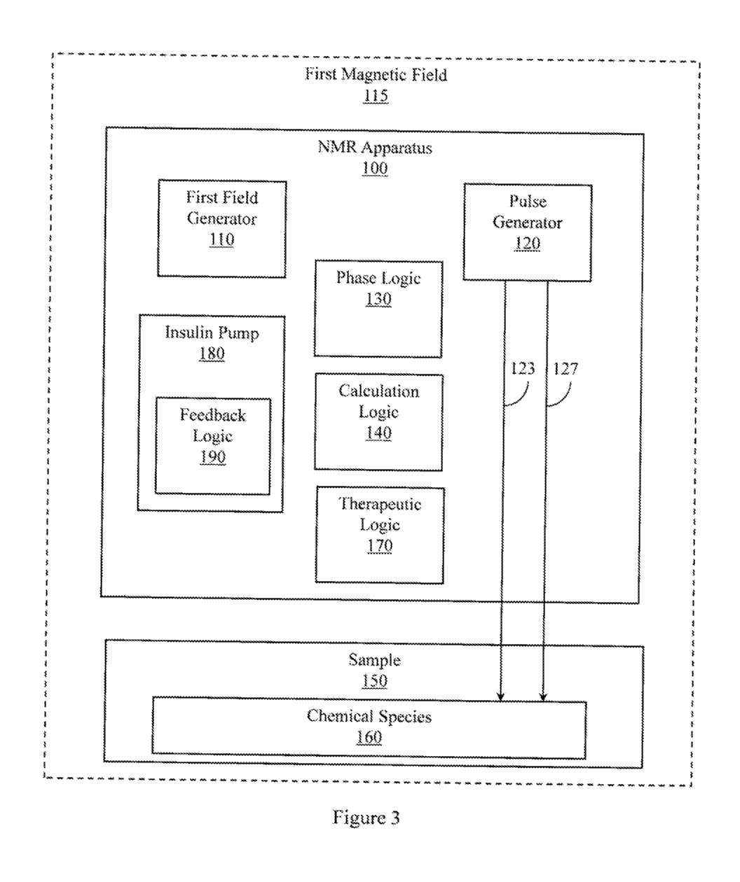

FIG. 3 illustrates an embodiment of an NMR apparatus 100 that is configured with an insulin pump 180 and a feedback logic 190. The insulin pump 180 administers to the patient the amount of insulin determined by the therapeutic logic 170. The feedback logic 190 adjusts the amount of insulin administered to the patient as a function of a change in the measurement of the amount of the chemical 160 species in the sample 150.

In one embodiment, the calculation logic 140 determines an amount of chemical species 160 (e.g., glucose) in a sample 150 (e.g., interstitial fluid, blood). A therapeutic logic 170 determines the amount of insulin to be administered to the patient based, at least in part, on the amount of chemical species 160 in the sample 150 as determined by relating an observed MR signal decay rate to known pairs (e.g., T2 decay rate to concentration, T1 decay rate to concentration, T2eff decay rate to concentration). In response to the therapeutic logic 170 determining the amount of insulin to be administered to the patient, the insulin pump 180 administers the amount of insulin to the patient. The feedback logic 190 will adjust the amount of insulin administered to the patient as the amount of chemical species 160 in the sample 150 changes.

Recall that the blood glucose levels of a diabetic patient vary throughout the day as a function of many variables (e.g., types of food consumed, when food is consumed, how much food is consumed, exercise habits). Therefore, patients must consistently monitor their glucose levels throughout the day and may need varying amounts of insulin based on their insulin level at any given time. The feedback logic 190 allows the insulin pump 180 to administer varying amounts of insulin as a function of a change in the measured amount of glucose in a blood sample.

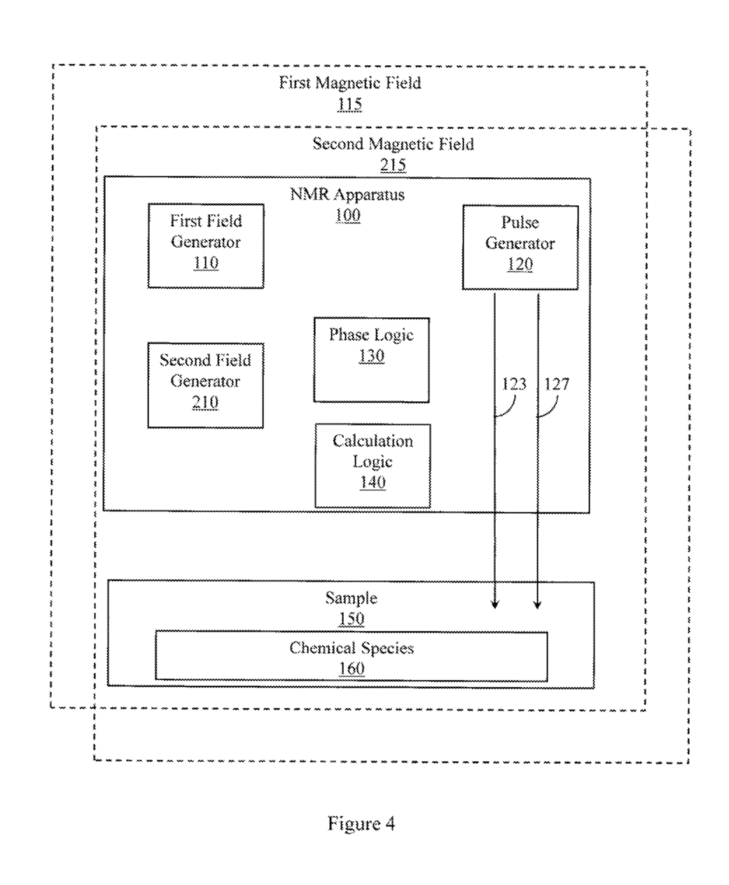

FIG. 4 illustrates an embodiment of an NMR apparatus 100 that is configured with a second field generator 210. The second field generator 210 is configured to apply a second magnetic field 215. The second magnetic field 215 may be spatially inhomogeneous, a pulsed field gradient, or a constant field. The first magnetic field 115 and the second magnetic field 215 are applied so that the first magnetic field 115 and the second magnetic field 215 can be applied to the sample 150.

In one embodiment, the second magnetic field 215 affects the pulse sequence applied by the pulse generator 120, in one embodiment, the NMR apparatus 100 may be used to measure the diffusion of the chemical species 160 in a sample 150. Accordingly, the pulse generator 120 may apply derivatives of the stimulated echo pulse sequence (PFG-STE). The pulse generator 120 may apply pulses in pairs. Specifically, the pairs may be bipolar pairs of gradient pulses to reduce artifacts in diffusion spectra.

In one embodiment, the pulse generator 120 provides the RF sequence a plurality of times. The NMR signal decay is measured by comparing a plurality of NMR signals acquired after the pulse generator 120 has provided the RF sequence a plurality of times.

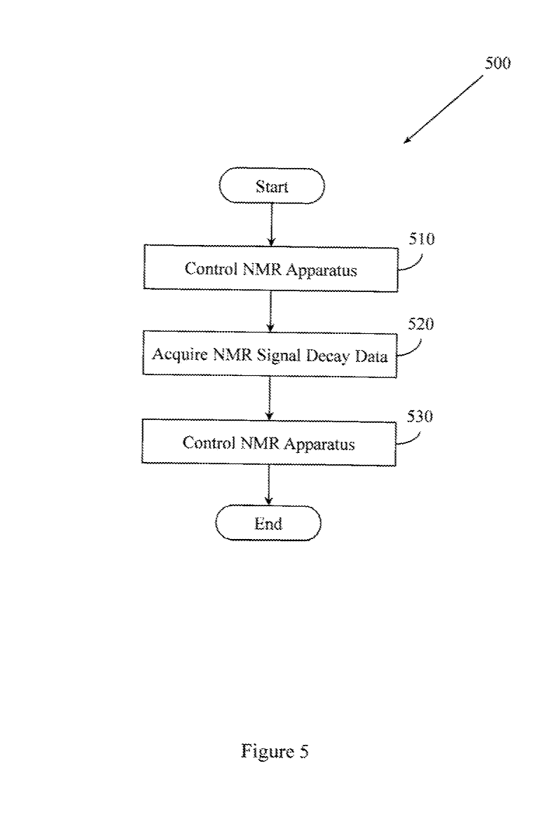

FIG. 5 illustrates a method associated with an NMR apparatus for determining the amount of a chemical species in a sample. The method 500 may be employed to determine the amount of glucose in a blood sample. Determining the amount of glucose in a blood sample may include determining the concentration of glucose in the sample.

Method 500 includes, at 510, controlling an NMR apparatus to apply a first magnetic field to a sample in a patient and to apply a RF pulse sequence to produce an NMR signal in nuclei associated with a chemical species in the sample.

Method 500 also includes, at 520, acquiring NMR signal decay data associated with a decay of the NMR signal produced in response to applying the first magnetic field and the RF signal.

Method 500 also includes, at 530, producing a characterization of a chemical species in the sample by comparing observed NMR signal decay data to previously acquired NMR signal decay data for known concentrations. One of ordinary skill in the art will appreciate that the characterization may include information about the chemical species in the sample including, but not limited to, the amount of the chemical species in the sample, the concentration of the chemical species in the sample, and the diffusion rate of the chemical species in the sample. The previously acquired NMR signal decay data may be available in forms including, for example, a table, a mathematical expression, a graph, or other form.

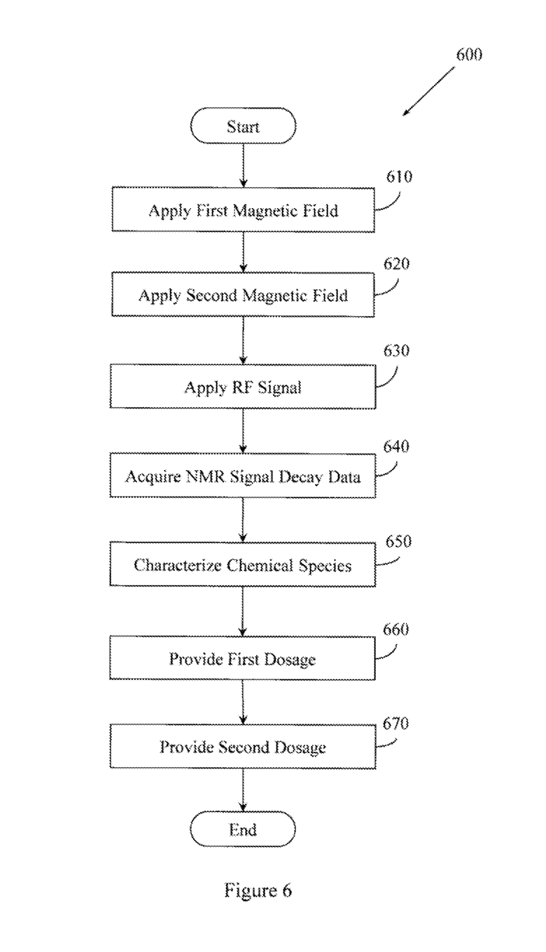

FIG. 6 illustrates a method 600 associated with of an NMR apparatus for determining the amount of a chemical species in a sample. Method 600 includes, at 610, controlling a NMR apparatus to apply a first magnetic field to a sample in a patient. The first magnetic field may be a static inhomogeneous applied magnetic field configured not to change in time.

Method 600 also includes, at 620, controlling the NMR apparatus to apply a second magnetic field to the sample. The second magnetic field may be a pulsed field gradient or a constant field gradient. In one embodiment, applying the first magnetic field at 610, and applying the second magnetic field at 620, is done in a manner that produces a spatially inhomogeneous field.

Method 600 also includes, at 630, applying an RF signal. The RF signal may be configured as a pulse sequence including a first RF pulse and a second RF pulse. The RF signal is configured to produce an NMR signal in nuclei associated with a chemical species in the sample located in the first magnetic field.

Method 600 also includes, at 640, acquiring NMR signal decay data associated with a decay of the NMR signal produced in response to applying the first magnetic field and the RF signal. The signal decay data may be used to discern information about the chemical species in the sample. For example, the NMR signal data may be compared to benchmark data that relates NMR signal decay rates to known concentrations of chemical species in a fluid.

Method 600 also includes, at 650, producing a characterization of a chemical species in the sample as a function of the NMR signal decay data. The characterization of a chemical species in the sample may be a measurement at a specific point in time or continually track the chemical species in the sample to measure changes in the chemical species in the sample (e.g., amount of chemical species in the sample).

Method 600 includes, at 660, controlling an insulin providing apparatus to provide a first dosage of insulin to the patient based, at least in part, on the amount of the chemical species in the sample.

Method 600 includes, at 670, controlling an insulin providing apparatus to provide a second different dosage of insulin to the patient, based at least in part, on a change in the characterization of the chemical species in the sample.

To the extent that the term “includes” or “including” is employed in the detailed description or the claims, it is intended to be inclusive in a manner similar to the term “comprising” as that term is interpreted when employed as a transitional word in a claim.

While example systems, methods, and so on have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A nuclear magnetic resonance (NMR) apparatus, comprising:
a memory that stores calibration data, where the calibration data relates known glucose concentrations in interstitial fluid to observed NMR signal decay rates;
a first field generator that provides a first magnetic field suitable for NMR;
a pulse generator that provides a radio frequency (RE) sequence at a frequency that produces an NMR signal in a sample located in the first magnetic field, where the sample is interstitial fluid;
a phase logic that measures NMR signal decay in the sample in vivo to produce a measured NMR signal decay, where the phase logic produces the measured NMR signal decay in place without touching the interstitial fluid, the interstitial fluid being located in a patient; and
a calculation logic that non-invasively and non-destructively determines an amount of glucose in the interstitial fluid by comparing the measured NMR signal decay to the calibration data.

2. The NMR apparatus of claim 1, comprising:
a therapeutic logic that continuously determines an amount of insulin to be administered to a patient based on the amount of glucose in the interstitial fluid.

3. The NMR apparatus of claim 2, comprising:
an insulin pump that administers the amount of insulin to the patient, where the insulin pump comprises a feedback logic that adjusts in real-time the amount of insulin administered to the patient based on a change in the measure of the amount of glucose in the interstitial fluid.

4. The NMR apparatus of claim 1, where the NMR apparatus is one of, mobile, wearable, and implantable.

5. The NMR apparatus of claim 1, where the first magnetic field is a static inhomogeneous applied magnetic field configured not to change in time.

6. The NMR apparatus of claim 1, where the pulse sequence comprises a first RF pulse to excite nuclei associated with glucose in the sample and a second RF pulse to cause the nuclei of the glucose to rephase according to their spatial position in the first magnetic field.

7. The NMR apparatus of claim 1, where the NMR signal decay is measured by comparing a plurality of NMR signals acquired after the pulse generator has provided the RF sequence a plurality of times.

8. The NMR apparatus of claim 1, where the NMR signal decay is described by:
S(t)=C*e^{-t/T2eff}
where S refers to the NMR signal,
C is a constant,
e is the exponential constant,
t refers to time, and
T2_{eff} refers to T2 effective.

9. A method, comprising:
accessing first data that correlates NMR signal decay rates to known concentrations of a chemical species in a fluid:
controlling a nuclear magnetic resonance (NMR) apparatus to apply a first magnetic field to a sample in a patient and to apply a radio frequency (RF) signal to produce an NMR signal in a fluid in the sample;
acquiring NMR signal decay data associated with a decay of the NMR signal produced by the fluid in response to applying the first magnetic field and the RF signal; and
producing a characterization of a chemical species in the sample in place without touching the sample by comparing the NMR signal decay data to the first data, where the decay rate of the NMR signal varies inversely with the concentration of the chemical species in the sample.

10. The method of claim 9, where the chemical species is glucose.

11. The method of claim 9, where the chemical species is a therapeutic compound.

12. The method of claim 9, where the chemical species is alcohol.

13. The method of claim 9, where the fluid is human interstitial fluid.

14. The method of claim 9, where the fluid is human blood.

15. The method of claim 9, where the first data is a table that relates NMR signal decay rates to known concentrations of a chemical species in a fluid.

16. The method of claim 9, where the first data is an expression that relates NMR signal decay rate to concentration of a chemical species in a fluid.

17. The method of claim 10, comprising:
controlling an insulin providing apparatus to provide a first dosage of insulin to the patient based, at least in part, on the concentration of the glucose in the sample.

18. The method of claim 17, comprising:
controlling the insulin providing apparatus to provide a second, different dosage of insulin to the patient based at least in part, on a change in the concentration of the glucose in the sample.

19. A method, comprising:
accessing data that describes a relation between a nuclear magnetic resonance (NMR) signal decay rate and a known concentration of glucose in a fluid;
acquiring, non-invasively and in-vivo, an observed NMR signal decay rate from a fluid in a patient, and
estimating a concentration of glucose in the fluid in the patient by comparing the observed NMR signal decay rate with the data that describes the relation between the NMR signal decay rate and the known concentration of glucose.

20. The method of claim 19, where the fluid is water and where the NMR signal decay rate is associated with T2_{eff}.
